(19) United States
(12) Patent Application Publication
(10) Pub. No.: US 2017/0350797 A1
(43) Pub. Date: Dec. 7, 2017

(54) Sample collection and DNA preservation device

(71) Applicants: Preston Estep, Weston, MA (US); Dominic Paratore, Manchester, MA (US)

(72) Inventors: Preston Estep, Weston, MA (US); Dominic Paratore, Manchester, MA (US)

(21) Appl. No.: 15/615,761

(22) Filed: Jun. 6, 2017

Related U.S. Application Data
(60) Provisional application No. 62/345,921, filed on Jun. 6, 2016.

(51) Int. Cl. G01N 1/28; C12Q 1/68; C12M 1/24; B01L 3/00; G01N 1/00; C12M 1/00

(52) U.S. Cl. CPC G01N 1/28; C12M 1/24; B01L 3/502; C12Q 1/68; B01L 3/50; C12M 1/00; G01N 2001/002

(57) ABSTRACT

The present invention relates to a biological sample collection kit, which uses a unique dry preservative for the preservation of nucleic acids, DNA and RNA, for collection, stabilization, transportation and long-term room temperature storage of biological samples that can be from sources such as saliva for molecular diagnostic applications. The invention features a collection device for biological samples such as saliva containing nucleic acids, and dry preservatives that are affixed directly or to a carrier substrate and placed within the collection device such that the samples come into contact with the dry preservatives resulting in the preservation and stabilization of the nucleic acids.

### CROSS-REFERENCE TO RELATED APPLICATION

This application claims the benefit of U.S. Provisional Application No. 62/345,921, filed Jun. 6, 2016, which is hereby incorporated in its entirety by reference.

### BACKGROUND OF THE INVENTION

The invention relates to a biological sample collection kit, including a sample collection device with dry preservatives for the collection, preservation and stabilization of nucleic acids from biological samples. For example, the sample collection device is useful for collecting and storing human saliva for DNA analysis.

Existing solutions for the collection and storage of biological samples suffer from several disadvantages. For the collection and storage of saliva samples, existing collection devices generally use preservative compounds in liquid form and comprising flammable substances such as ethanol. Disadvantages of these devices include: preservative liquids can leak as the devices are transported or shipped to end users; it is counterintuitive for end users to expect the collection device to already contain a liquid prior to collection, which can lead to accidental spills; even if the end user is aware of the presence of preservative liquids they can be spilled accidentally during collection and handling of the collection device; flammable substances like ethanol complicate the manufacturing process for the insertion of the liquid in a safe and practical way, and introduce additional risks when shipping, transporting or storing existing collection devices; liquid preservatives add liquid volume to the saliva that needs to be removed during analysis; the additional liquid volume also requires a larger size collection device, and introduces additional weight during shipment and transportation. In addition, existing devices typically present long-term storage challenges, particularly if a portion of the liquid preservative has been spilled or has not been adequately mixed in with the collected sample.

Some devices attempt to address these disadvantages by sequestering the liquid preservative within a sealed applicator that is easier to use, but these devices do not overcome the disadvantages inherent to liquid preservatives during transportation, shipment, analysis and storage, including increased volumes and spill or flammability risks. Other attempted solutions seek to seal the liquid into a special self-dosing cap or lid, but these solutions also fail to meet industry needs because they are counterintuitive and can still result in spills, and also fail to address increased volumes and flammability risks.

Examples of sample collection devices are found in the following patents and patent applications: U.S. Pat. No. 7,482,116, U.S. Pat. No. 8,221,381, U.S. Pat. No. 8,728,414, U.S. Pat. No. 9,207,164, U.S. Ser. No. 13/333,883 and U.S. Ser. No. 14/448,721.

There is a need for a user-friendly sample collection device that allows even inexperienced users to easily and reliably collect, store and transport biological samples, such as saliva, for DNA analysis in over-the-counter or research settings while avoiding the risks and disadvantages of liquid sample preservatives. Furthermore, it would be desirable to have a device that is safe and easy to use in over-the-counter or home-based applications and that protects end users from accidental spills and accidental contact with the preservative. Still further, it would be desirable to have a device that isn't rated as flammable, as is typical of devices that include flammable substances such as ethanol in liquid preservatives. Therefore, there currently exists a need for a device and associated method that is easy to use and uses dry preservative chemistry to avoid the disadvantages of liquid chemistry preservatives.

### BRIEF SUMMARY OF THE INVENTION

The present invention overcomes the aforementioned disadvantages by providing a collection kit including a collection device with one or more dry preservatives. The device allows easy and reliable collection, preservation and stabilization of nucleic acids in biological samples, such as saliva, in over- the-counter or research settings and avoids the use of liquid chemistry for the preservative, facilitating the safe transport, storage and use of the collection kit and collection device.

The present invention is a biological collection and preservation kit including a collection device that can collect and preserve nucleic acids DNA and RNA from biological samples such as saliva. The collection device comprises the following components: an optional mechanism, such as a funnel, to facilitate collection of the saliva (sample); a container such as a tube, vial or vessel to contain the sample; one or more dry preservatives to preserve and stabilize the sample; and a mechanism to seal the container, such as a cap that creates a water tight seal. These components are connected as follows: the dry preservatives are affixed to the interior of the container or the interior-facing portion of the sealing mechanism or cap, either directly or on a substrate; the sample, such as saliva, is collected into the container via a direct or indirect interface device such as a funnel or absorbent collection applicator; and the container is sealed using the sealing mechanism.

The collection kit of the present invention may also include one or more of the following: a barcode to uniquely serialize the device and which may be used to track the source of the sample; an RF identifier that uniquely serializes the device and can be used to track the source of the sample; and a container design that facilitates robotic sample processing following saliva collection. Similarly, the method associated with the present invention may also include easy application of DNA or RNA precipitate chemistry to facilitate the processing of the sample for molecular diagnostic applications.

The present invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

FIG. 1 depicts an embodiment of the sample collection device according to the invention, comprising: a tube, a cap, an optional funnel, a dry preservative affixed directly to the interior of the tube at the bottom, and a dry preservative on a substrate affixed to the interior cylindrical surface of the tube.

### DETAILED DESCRIPTION OF THE INVENTION

The present invention is directed to biological collection and preservation kits including a collection device that can collect and preserve nucleic acids DNA and RNA from biological samples such as saliva.

An embodiment of a saliva collection kit according to the invention may comprise the following: a collection interface 40 that may be a saliva funnel; a tube, vial or vessel 30 for the collection of the saliva sample; dry preservatives 10, 20 stored within the tube, vial or vessel 30 that will preserve and stabilize the nucleic acids DNA and RNA in the saliva on contact; a sealing mechanism 50 for the tube, vial or vessel such as a cap; unique serialization information in barcode and/or RF ID form to identify the kit and the source of the saliva; an optional mechanism for recording user information on the tube, vial or vessel; an optional mechanism to assist in the application of pre-analysis chemistry; an end-user delivery container to hold and deliver the aforementioned components for use along with instructions; and a return container to provide for storage and shipping of collected samples to a lab for processing.

The components of the kit are interconnected as follows. The end-user delivery container for the kit contains the instructions, the collection tube, vial or vessel 30 with a pre-attached saliva collection interface 40 (such as a funnel) and the sealing mechanism 50 or cap. The unique identifying barcode or RF ID is permanently embedded in or attached to the tube, vial or vessel 30 during manufacturing or prior to delivery to the end-user. The end-user delivery container may be reused as a return container, or a return container may be provided with it. The return container may incorporate a biohazard bag or other leak-proof means of enclosing the tube, vial or vessel containing biological samples. A return shipping label and/or prepaid postage may also be included with the end-user delivery container for use with the return container. Information identifying the end user or the sample can be directly recorded onto the tube, vial, or vessel at the time of collection. One or more dry preservative substances or compounds 10, 20 are applied to the inside of the tube, vial or vessel 30 such that they become affixed and won't spill out. The preservatives 10, 20 may be applied directly to the interior surface of the tube or cap, or they may be applied on to a substrate that is then affixed within the tube 30, or a combination of these approaches may be used. The dry preservative may be applied to the surface of the substrate, tube, vial or vessel as a liquid which then is dried into place. The preservatives 10, 20 are preferably located inside the tube 30 such that they come into contact as soon as possible with the collected saliva.

In one embodiment of the invention the dry preservative comprises a blend of reagents and enzymes that allow for complete lysis and stabilization of the nucleic acids DNA and RNA and easy transportation and long-term storage of samples at room temperature prior to genetic analysis. The key ingredients of the preservatives have the following functions: Ingredient A—denatures enzymes, including nucleases, including DNase enzymes; Ingredient B—chelates metal cofactors required by DNase enzymes; Ingredient C—denatures enzymes and helps reduce viscosity of saliva along with other supporting chemicals; Ingredient D—hydration agent; Ingredient E—a pH buffer to ensure a desired pH is achieved in the resulting preservative and to contribute to DNA and RNA precipitation; Ingredient F—encourages DNA and RNA precipitation; Ingredient G—modifies the sample's viscosity and surface tension; Ingredient H—digests nucleases in the sample. In one embodiment, the ingredients are combined to form two separate dry preservatives. The first preservative 10 comprises ingredients A through F and may also comprise ingredient G. The second preservative 20 comprises ingredient H and may also comprise ingredient F and/or G.

Ingredient A may be a substance such as Sodium Dodecyl Sulfate or similar. Ingredient B may be a substance such as CDTA, 1,2-CyclohexaneDiamineTetraAcetic Acid or similar. Ingredient C may be a substance such as urea or similar. Ingredient D may be a substance such as 1,2-Propanediol or similar. Ingredient E may be a substance such as NaOAc/Tris or similar. Ingredient F may be a substance such as Polyethylene Glycol 8000 or similar. Ingredient G may be a surfactant such as Hexadecyltrimethylammonium Chloride or similar. Ingredient H may be a substance such as Proteinase K or similar.

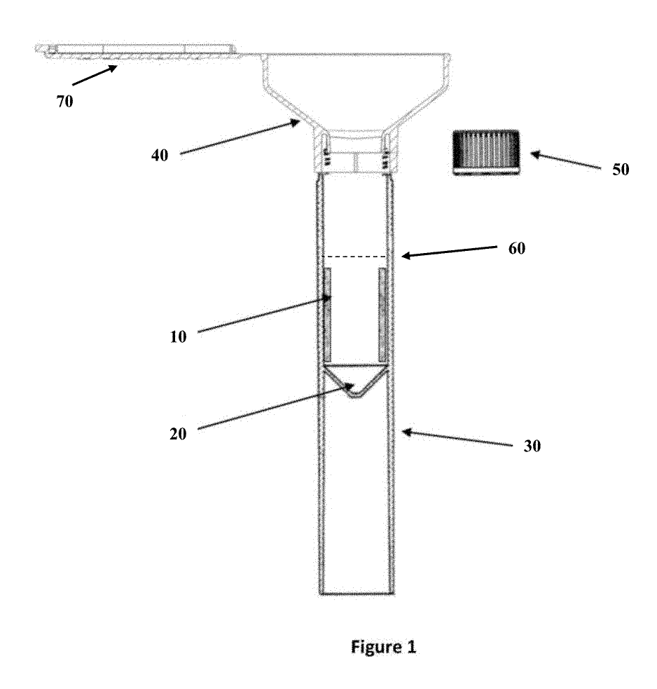

FIG. 1 depicts a sample collection device according to the invention. The device is intended for use in the non-invasive collection of saliva samples. DNA stabilized (preserved) using the device may be used for molecular diagnostic applications and over-the-counter DNA carrier screening genotyping tests. Item 30 is a tube or vial for containing the collected saliva sample. Item 50 is cap that will allow for the closure of the vial 30 such that it won't leak. Item 20 is the second dry preservative described above, which is shown as coated onto the bottom of the vial, but it may be coated onto other locations as well, such as the interior of cap 50. Item 10 indicates the first dry preservative described above, which is depicted as coated onto a carrier substrate, but it may be directly coated onto the inside of the vial 30. The substrate containing the first dry preservative 10 may be paper, plastic or a similar material. Item 40 is a funnel, shown including a press-fit cap 70, that facilitates collection of saliva into the vial 30. Funnel 40 may be permanently attached to vial 30 or it may be removable, as shown. Once funnel 40 has been used to direct saliva into the vial 30 it is removed and cap 50 is used to seal the vial 30. Item 60 indicates the location of a fill-line that provides the user a visual indication that sufficient saliva has been collected in vial 30.

In one embodiment the vial 30 is made of polypropylene, has a capacity of 5 mL, and the fill line 60 is at 3 mL. The carrier substrate coated with the first preservative may be a piece of chromatography paper, 43×20 mm, and may be held in position by a silicone retention ring. The second preservative may comprise a dried solution of Proteinase K, and may also comprise Polyethylene Glycol 8000 and a surfactant. The first preservative may comprise a dried solution of the following ingredients: Sodium Dodecyl Sulfate; 1, 2-CyclohexaneDiamineTetraAcetic Acid; Urea; NaOAc/Tris (stock: 2M NaOAc, 0.5M Tris, PH8.0); Polyethylene Glycol 8000; 1,2-Propanediol, Sodium Hydroxide (to adjust pH) and may also comprise a surfactant such as, for example, Hexadecyltrimethylammonium Chloride.

A method according to the invention may comprise the following steps: 1. Unbox kit from initial shipping container; 2. Remove kit package (plastic thermoform or similar); 3. Open kit package and remove sample collection tube 30, which includes a pre-attached funnel 40; 4. Open the funnel 40 if necessary (the funnel may feature a flip top cap 70) and spit or drool into the funnel to collect the saliva and fill the tube 30 until the collected saliva, not counting bubbles or foam, reaches the fill line 60; 5. Remove the funnel 40, if removable, and discard it; 6. Seal the tube 30 using the included cap 50; 7. Invert the vessel to accelerate mixing of the saliva with the dry preservatives 10 and 20; 8. Record user or sample identifying information onto the tube if desired; 9. Place the sealed tube 30 into the biohazard bag or other leak-proof storage or shipping enclosure; 10. The sample is ready for processing or it can be placed into the shipping package, should one have been provided, for storage and/or laboratory processing; 11. Upon processing the pre-analysis feature can be utilized if the collection device is so equipped; 12. Sample process and analysis is conducted via the recommended nucleic acid (DNA and RNA) isolation process and then carried out via the laboratory's protocol.

All references, issued patents and patent applications cited within the body of the instant specification are hereby incorporated by reference in their entirety, for all purposes. While the invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims.

What is claimed is:

1. A saliva collection device for preserving nucleic acids, comprising:
a container having an interior surface;
a first dry preservative affixed onto a first portion of the interior surface of said container;
a second dry preservative affixed onto a second portion of the interior surface of said container; and
a cap.

2. The device of claim 1, wherein the first dry preservative is a compound comprising a denaturing agent.

3. The device of claim 2, wherein the compound further comprises a chelating agent.

4. The device of claim 3, wherein the compound further comprises a pH buffer.

5. The device of claim 1, wherein the first dry preservative is a compound comprising sodium dodecyl sulfate.

6. The device of claim 5, wherein the compound further comprises CDTA, 1, 2-CyclohexaneDiamineTetraAcetic Acid.

7. The device of claim 6, wherein the compound further comprises urea.

8. The device of claim 7, wherein the compound further comprises NaOAc/Tris.

9. The device of claim 8, wherein the compound further comprises Polyethylene Glycol 8000.

10. The device of claim 9, wherein the compound further comprises 1,2-Propanediol.

11. The device of claim 1, wherein the second dry preservative comprises a protease.

12. The device of claim 1, wherein the second dry preservative comprises Proteinase K.

13. The device of claim 1, wherein the container is a tube.

14. The device of claim 1, wherein the container is a vial.

15. The device of claim 1, wherein the first dry preservative is included in a substrate, said substrate being affixed to said first portion of the interior surface of said container.

16. The device of claim 15, wherein the substrate is paper.
